(19) United States
(12) Patent Application Publication
(10) Pub. No.: US 2023/0248393 A1
(43) Pub. Date: Aug. 10, 2023

(54) EXTERNAL BONE FIXATION DEVICE WITH MODULAR DRIVING ELEMENT

(71) Applicant: Nuvasive Specialized Orthopedics, Inc., San Diego, CA (US)

(72) Inventors: Shanbao Cheng, Rancho Santa Margarita, CA (US); Shawn Placie, Aliso Viejo, CA (US); Michael Moeller, Aliso Viejo, CA (US)

(21) Appl. No.: 18/106,729

(22) Filed: Feb. 7, 2023

Related U.S. Application Data
(60) Provisional application No. 63/308,905, filed on Feb. 10, 2022.

(51) Int. Cl. A61B 17/62; A61B 17/66

(52) U.S. Cl. CPC A61B 17/62; A61B 17/66; A61B 90/06

(57) ABSTRACT

Various implementations include bone fixation devices and related methods. Certain implementations include an external bone fixation device that includes a modular driving element for selectively coupling to an adjustment element and controlling adjustment of one or more struts.

### CROSS REFERENCE TO RELATED APPLICATION

The present patent application claims priority to U.S. provisional patent application No. 63/308,905, filed Feb. 10, 2022, the entirety of which is incorporated by reference as though fully set forth herein.

### TECHNICAL FIELD

This disclosure generally relates to bone fixators. More particularly, the disclosure relates to the field of external bone fixators.

### BACKGROUND

External bone deformity correction devices or bone adjustment systems (referred to as “external bone fixation devices” or “external fixators” herein) conventionally include fixation components, frames, or rings, and adjustable bodies (e.g., interconnecting bodies or struts) for building and/or adjusting the devices. These components can be used intraoperatively (e.g., to build a fixation device) or postoperatively (e.g., to adjust the fixation device). However, conventional fixation devices may lack adaptability, for example in use by both medical professionals and patients.

### SUMMARY

The needs above, as well as others, are addressed by embodiments of bone fixation devices, and related methods described in this disclosure. All examples and features mentioned below can be combined in any technically possible way.

Various implementations include bone fixation devices and related methods. Certain implementations include an external bone fixation device that includes a modular driving element for selectively coupling to an adjustment element and controlling adjustment of one or more struts.

In particular aspects, a bone fixation device for attaching to a bone externally to a body includes: a set of two or more rings, each ring in the set of rings being coupled with distinct portions of the bone via corresponding bone connectors; a strut spanning between two of the rings in the set of rings; an adjustment element for adjusting a length of the strut; and a modular driving element for selectively coupling to the adjustment element and controlling adjustment of the length of the strut.

In additional particular aspects, a method includes adjusting a bone fixation device attached to a bone external to a body. The bone fixation device has: a set of at least two rings coupled with distinct portions of the bone via corresponding bone connectors; at least one strut spanning between two of the rings in the set of rings; and an adjustment element for adjusting a length of the strut. The method includes: coupling a modular driving element or a manual adjustment tool to the adjustment element; and actuating adjustment of the length of the strut with the modular driving element or the manual adjustment tool.

In further particular aspects, a method includes imaging a bone connected with a bone fixation device external to a body. The bone fixation device has: a set of at least two rings coupled with distinct portions of the bone via corresponding bone connectors; at least one strut spanning between two of the rings in the set of rings; an adjustment element for adjusting a length of the strut; and a modular driving element coupled to the adjustment element. The method includes: decoupling the modular driving element from the adjustment element; and imaging the bone with at least one of MRI or X-ray imaging.

Implementations may include one of the following features, or any combination thereof.

In particular aspects, the modular driving element is configured to actuate the adjustment element to control adjustment of the length of the strut.

In certain cases, the adjustment element is fixed to the strut and the modular driving element is couplable and decouplable from the adjustment element.

In particular cases, the modular driving element can be coupled and decoupled from the adjustment element without a tool.

In some aspects, the bone fixation device further includes a plurality of additional struts spanning between the two rings, and each of the plurality of additional struts includes a distinct adjustment element for adjusting a length of the corresponding strut.

In certain implementations, the bone fixation device further includes a plurality of additional modular driving elements for selectively coupling to each of the distinct adjustment elements to control adjustment of the length of each corresponding strut.

In particular aspects, each of the modular driving elements is individually programmable to control an amount of the adjustment of the length of the corresponding strut.

In some cases, at least one of the modular driving elements is configured to adjust the length of the corresponding strut without the presence of all of the modular driving elements.

In certain aspects, each of the modular driving elements has a separate power source.

In particular cases, at least two of the modular driving elements share a common power source.

In some implementations, the set of rings includes at least three rings.

In certain cases, the adjustment element includes a linear actuator.

In particular implementations, the modular driving element is wirelessly coupled with a controller for controlling adjustment of the length of the strut or is wirelessly coupled with the controller for controlling adjustment of the length of the strut.

In some aspects, the controller includes a remote control device dedicated to controlling adjustment of the length of the strut or a smart device configured to operate a control platform for adjusting the length of the strut.

In certain implementations, the controller is configured to run as a control platform at a remote location relative to the bone fixation device.

In particular aspects, the adjustment element and the modular driving element each comprise a complementary linkage configured to interface with one another and enable adjusting the length of the strut.

In some cases, the adjustment element and the modular driving element include magnets configured to interface with one another and enable adjusting the length of the strut. In particular aspects, the magnets enable adjustment of the length of the strut without physical contact between the adjustment element and the modular driving element.

In particular implementations, the modular driving element includes: a driving actuator for interfacing with a driven mechanism on the adjustment element; a control unit for controlling actuation of the driving actuator; and a communication system coupled with the control unit, the communication system configured to receive instructions to actuate the driving actuator. In certain aspects, the driving actuator includes at least one of a gear-and-motor system, magnets, or pneumatics. In some aspects, the communication system is configured to communicate over at least one of: Bluetooth, Bluetooth Low Energy (BLE), radio frequency (RF), Wi-Fi, ultrasound, or an independent SIM card. In particular cases, the communication system can send and/or receive data from a cloud storage system.

In some aspects, the bone fixation device further includes a motor coupled with the control unit at the driving actuator.

In particular cases, the bone fixation device further includes a power source coupled with the control unit and the driving actuator, where an output of the power source is prescribed according to an amount of potential adjustment of the length of the strut. In certain cases, power sources of different capacities (e.g., different power storage capacities) can enable distinct adjustment amounts for distinct struts.

In certain implementations, the adjustment element is embedded in the strut. In particular cases, the adjustment element is hermetically sealed in the strut with a power source such as a battery.

In particular aspects, the bone fixation device provides negligible distortion of imaging of the bone under both X-ray imaging and MM.

In some cases, the bone fixation device further includes a feedback system in communication with the modular driving element, where the modular driving element provides feedback on a force response to the adjustment of the length of the strut.

In certain aspects, the feedback system includes a sensor. In some implementations, the sensor includes a load cell, a piezoelectric sensor, a position sensor, a speed sensor, a gyroscope, a temperature sensor, a humidity sensor and/or an imaging sensor such as ultrasound.

In particular cases, the feedback system provides instructions to a controller for the modular driving element to modify a force applied during the adjustment of the length of the strut based on the feedback on the force response.

In some aspects, the feedback system includes a model correlating force response and force applied during adjustment of the length of the strut, where the model is based at least in part on historical data from a set of struts in distinct bone fixation devices.

In certain implementations, the bone fixation device further includes a manual adjustment tool for mating with the adjustment element to adjust the length of the strut while the modular driving element is decoupled from the adjustment element.

In particular cases, the bone connectors include at least one of bone screws or wires.

In some aspects, the modular driving element includes a self-powered actuator.

In certain cases, a method further includes: decoupling the coupled modular driving element or manual adjustment tool from the adjustment element; coupling the other one of the modular driving element or the manual adjustment tool to the adjustment element; and actuating adjustment of the length of the strut.

In particular implementations, a method further includes: for the modular driving element: receiving feedback about a force applied in actuating the adjustment of the length of the strut; and adjusting the force applied in actuating adjustment of the length of the strut in response to the feedback indicating that the force applied in actuating adjustment of the length of the strut deviates from a threshold.

In certain aspects, when the modular driving element is decoupled from the adjustment element, the bone fixation device provides negligible distortion of imaging of the bone under Mill and X-ray imaging.

In particular cases, a method further includes coupling the modular driving element to the adjustment element or coupling a manual adjustment tool to the adjustment element after imaging the bone.

Two or more features described in this disclosure, including those described in this summary section, may be combined to form implementations not specifically described herein.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

The details of one or more implementations are set forth in the accompanying drawings and the description below. Other features, objects and benefits will be apparent from the description and drawings, and from the claims.

### BRIEF DESCRIPTION OF THE DRAWINGS

FIG. 1 shows a perspective view of a bone fixator coupled with a bone according to various implementations.

FIG. 2 shows a perspective view of portions of a bone fixator according to various implementations.

FIG. 3, FIG. 4, and FIG. 5 show distinct perspective views of a bone fixator according to various implementations.

FIG. 6 shows a perspective view of a portion of a bone fixator according to various implementations.

FIG. 7 shows a perspective view of a portion of a bone fixator according to various additional implementations.

FIG. 8 shows a perspective view of a portion of a bone fixator according to various additional implementations.

FIG. 9 is a perspective view of a portion of a bone fixator according to various implementations.

FIG. 10 is a perspective view of a modular driving element according to various further implementations.

It is noted that the drawings of the various implementations are not necessarily to scale. The drawings are intended to depict only typical aspects of the disclosure, and therefore should not be considered as limiting the scope of the implementations. In the drawings, like numbering represents like elements between the drawings.

### DETAILED DESCRIPTION

Various example embodiments of devices and techniques for adjusting and/or imaging a bone fixation device are described herein. In the interest of clarity, not all features of an actual implementation are necessarily described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The bone fixation devices and related systems, program products and methods described herein boast a variety of inventive features and components that warrant patent protection, both individually and in combination.

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

This disclosure provides, at least in part, an external bone fixation device and methods that beneficially incorporate a modular driving element to enhance patient engagement, medical professional adjustment, and/or imaging. The various disclosed implementations can improve patient outcomes when compared with conventional external bone fixation devices. The disclosed implementations can provide modularity in adjusting a bone fixation device, enhancing both intraoperative and postoperative engagement with the device. In particular cases, the bone fixation device can provide a modular driving element for selectively engaging with an adjustment element to control adjustment of one or more device struts. The modularity of the driving element/adjustment element coupling can enable efficient transition from operative scenarios (e.g., including imaging during or post-operatively) to post-operative scenarios (e.g., with at-home adjustment by a patient and/or remote adjustment by a medical professional). The driving elements can include one or more mechanical and/or electro-mechanical components, and in certain cases, are configured to communicate with one or more remote systems to control device adjustment and/or exchange data about adjustments.

Commonly labeled components in the FIGURES are considered to be substantially equivalent components for the purposes of illustration, and redundant discussion of those components is omitted for clarity.

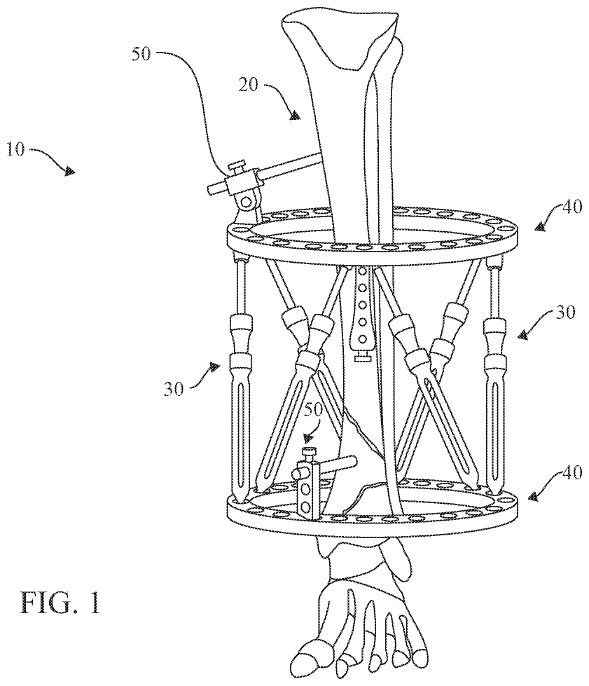

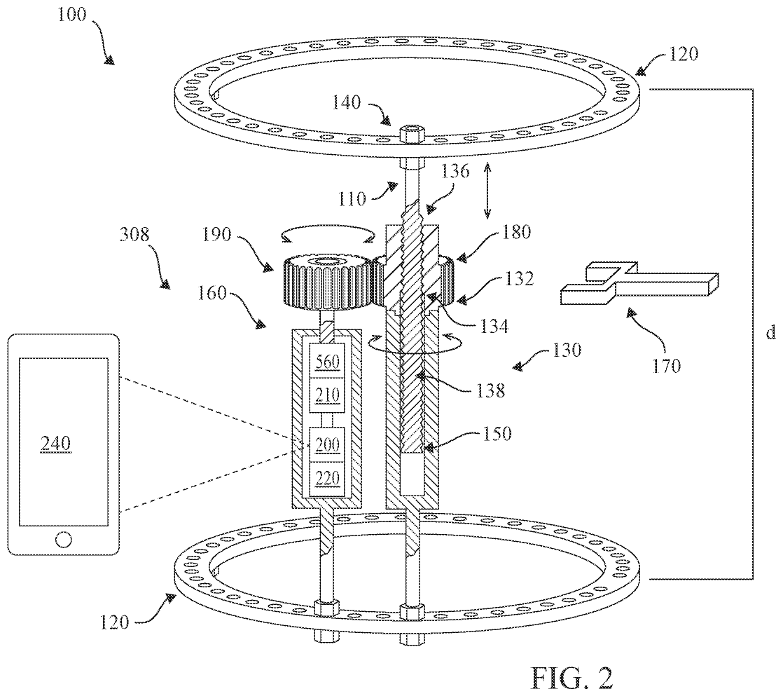

FIG. 1 shows an example of a bone fixation device 10 for attaching to a bone 20. A set of struts 30 are shown spanning between two rings 40. Bone connectors 50 are coupled to the rings and to the bone 20 for actuating adjustment of the bone 20. In various implementations, bone connectors 50 include bone screws and/or wires for anchoring in a patient's bone 20.

FIG. 2 is a perspective view of a particular bone fixation device 100, separated from a bone 20, according to various particular implementations. As with bone fixation device 10, the bone fixation device 100 is configured to attach to a bone external to a patient's body, that is, external to the patient's skin. Bone fixation device 100 is illustrated in this example as having only a single strut 110 between rings 120 (two shown, in this example). However, it is understood that a plurality of struts 110 can span between any two adjacent rings 120 in the device 100. For example, four (4) or more struts 110 can be located annularly around the patient's bone between any two adjacent rings 120. It is further understood that the bone fixation device (or simply, device) 100 can include a plurality of rings 120, e.g., three or more rings 120 with struts 110 spanning between. It is further understood that bone connectors similar to the bone connectors 50 illustrated in FIG. 1 can be coupled with the rings 120 to connect with the patient's bone(s).

FIG. 2 also illustrates an adjustment element 130 for adjusting a length of the strut 110, which thereby adjusts the distance between the adjacent rings 120 joined by the strut 110. In certain cases, the adjustment element 130 spans at least a portion of the distance (d) between the adjacent rings 120, such that the adjustment element 130 is able to envelop, overlap, or otherwise integrate a portion of the length of the strut 110 to perform a length adjustment. That is, in certain implementations, a first end 140 of the strut 110 is coupled to a ring 120, and a second end 150 of the strut 110 (opposing first end 140) is coupled to the adjustment element 130. In this sense, the strut 110 and the adjustment element 130 can be integrated to collectively define the distance (d) between adjacent rings 120. In particular implementations, the adjustment element 130 is fixed to the strut 110. In other implementations, the adjustment element 130 is removable from the strut 110, e.g., by hand, to enable substitution of distinct adjustment element(s) for connecting with the strut 110. As illustrated in FIG. 2, in some cases, the adjustment element 130 includes a linear actuator 132 for adjusting the length of the strut 110. In certain aspects, the linear actuator 132 includes a linkage 134 with a corresponding (e.g., complementary) linkage 136 on the shaft 138 of the strut 110. As the adjustment element 130 is actuated, the linear actuator 132 can translate, rotate, or otherwise move to initiate movement of the corresponding linkage 136 on the shaft 138. In the example illustrated in FIG. 2, the linear actuator 132 includes a set of teeth (e.g., gears) for engaging with teeth on the shaft 138 of the strut 110. As the linear actuator 132 moves, so do the teeth in linkages 136 and 138, thereby driving the shaft 138 to displace rings 120 relative to one another. It is understood that the gear/teeth-based linkage illustrated in the example of FIG. 2 can be replaced with any suitable linear actuator described and/or illustrated herein.

In various implementations, a modular driving element 160 is selectively couplable to the adjustment element 130, for example, to control adjustment of the length of the strut 110. That is, the modular driving element 160 can be coupled with, and decoupled from, the adjustment element 130 to actuate the adjustment element 130 for controlling adjustment of the length of the strut 110. In certain cases, the modular driving element 160 can be coupled with and decoupled from the adjustment element 130 by hand, e.g., without a tool. In other cases, the modular driving element 160 can be coupled and decoupled from the adjustment element 130 with a commonly available tool such as an Allen Key. As described herein, the modular driving element 160 can be programmatically controllable, e.g., by an electronic controller, programmable computing device, printed circuit board, etc. In this sense, the modular driving element 160 is configured to drive the adjustment element 130 with a powered (e.g., electrically powered) mechanism. In various implementations, the modular driving element 160 and the adjustment element 130 have a complementary linkage configured to interface with each other and enable adjusting the length of the strut 110. For example, the modular driving element 160 and the adjustment element 130 can include complementary mechanical components such as interlocking gears, tabs or slots. As described herein, these complementary mechanical components can be substituted for magnetic (non-contacting) components configured to interface with one another and enable adjustment of the strut 110 in certain cases.

In various implementations, the adjustment element 130 can also be actuated by a manual adjustment tool 170. In certain cases, the manual adjustment tool 170 is configured to mate with the adjustment element 130 to adjust the length of the strut 110 while the modular driving element 160 is decoupled from the adjustment element 130. That is, the manual adjustment tool 170 is an alternative mechanism to the modular driving element 160 for actuating the adjustment element 130, e.g., to adjust the length of the strut 110. In the various implementations described herein, the manual adjustment tool 170 and the modular driving element 160 can be configured to engage with the adjustment element 130, e.g., via a mount, clasp, lock or other coupler (not shown). In some cases, the mount (or other coupler) is coupled with the adjustment element 130 and/or one of the rings 120 to position the modular driving element 160 or the manual adjustment tool 170 adjacent to the adjustment element 130.

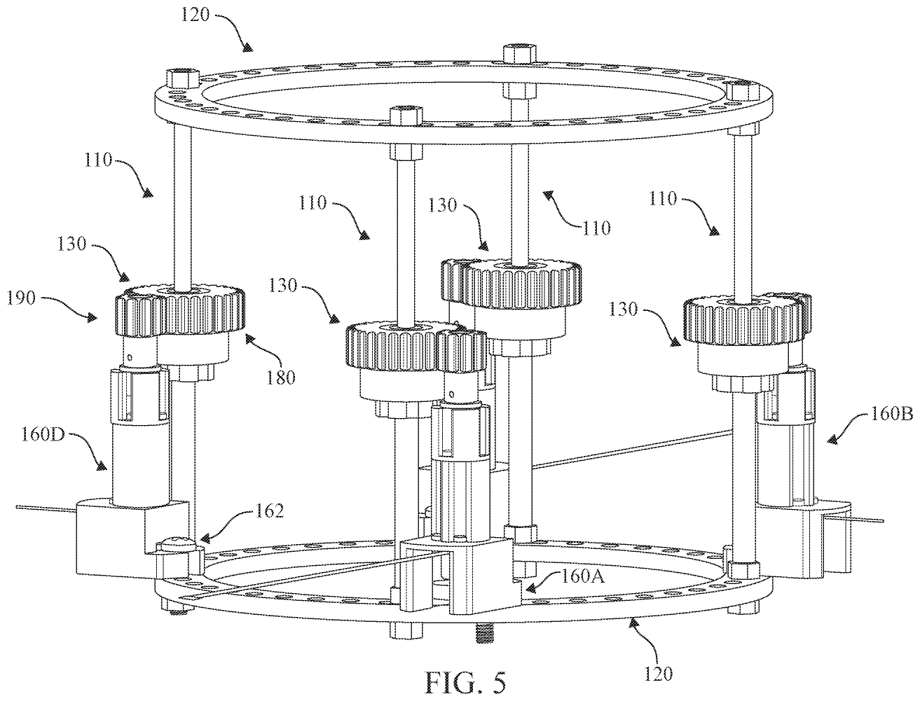

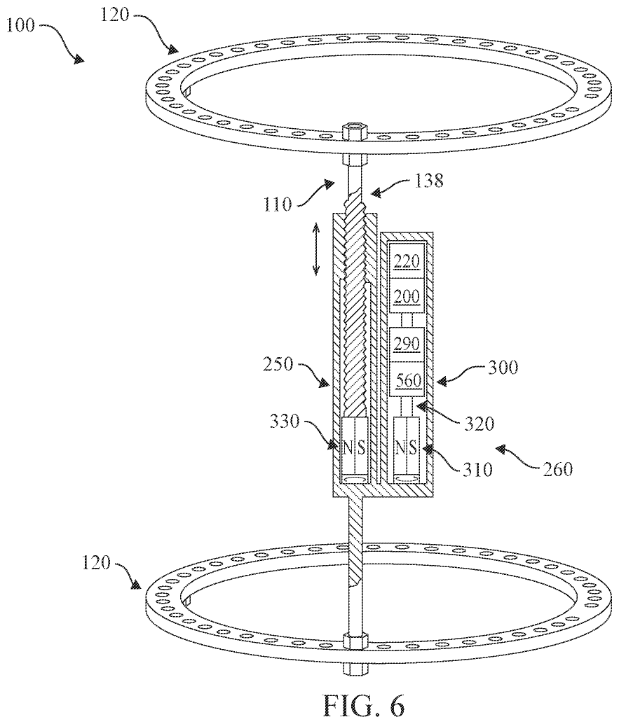

According to various implementations, the manual adjustment tool 170 is configured to complement a driven mechanism 180 on the adjustment element 130. In the example depicted in FIG. 2, the driven mechanism 180 includes at least one gear (or other interlocking member configuration) with teeth for interfacing with corresponding teeth on the manual adjustment tool 170. However, in other configurations, the manual adjustment tool 170 can include a clamp, vice, grip, adhesive, etc., that can be connected with the driven mechanism 180 to manually drive the adjustment element 130. As described herein, the manual adjustment tool 170 and driven mechanism 180 can include complementary actuators for driving the adjustment element 130. The implementation shown in FIG. 2 can include mechanical manual adjustment tool(s) 170 such as, but not limited to: a wrench, a screwdriver, pliers, an Allen tool (e.g., key), etc. Additional adjustment mechanisms, including for example, additional adjustment elements and manual adjustment tools utilizing magnets are illustrated and described with reference to various additional implementations (e.g., in FIGS. 6-11).

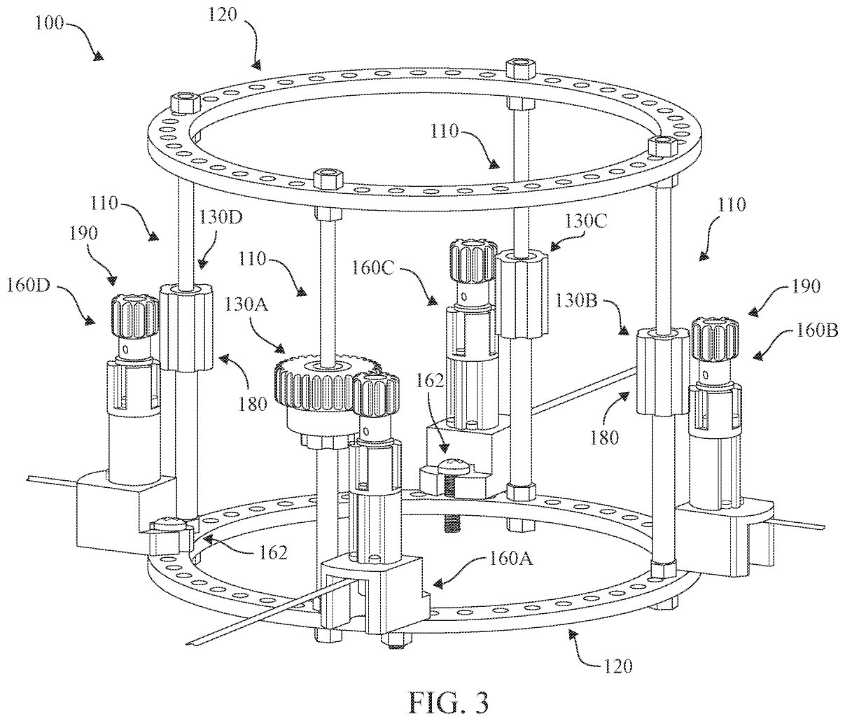

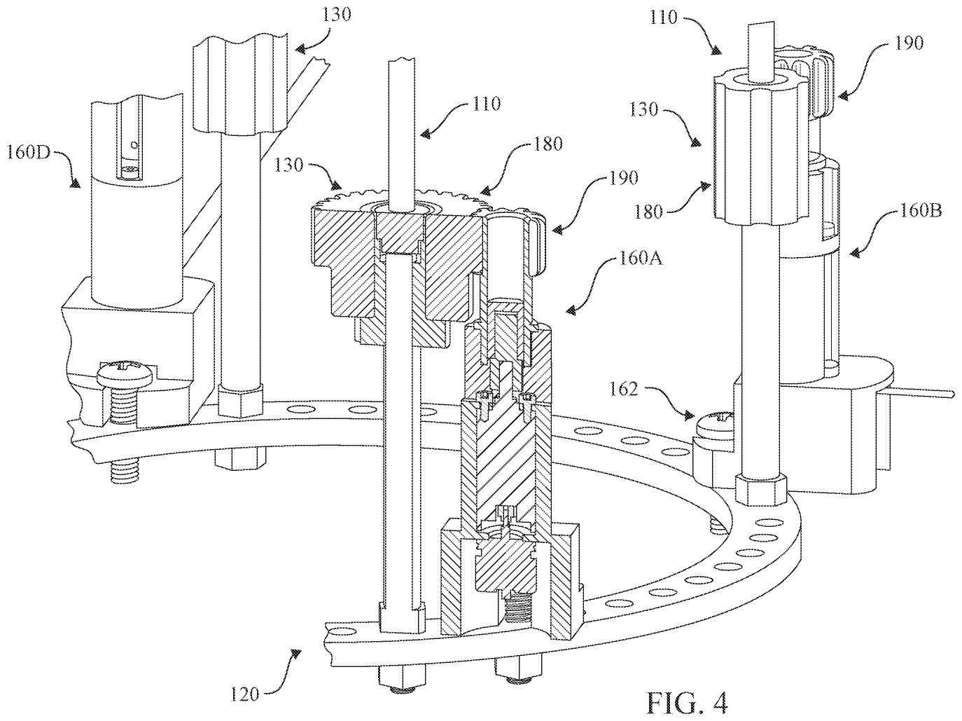

As noted herein, a plurality of struts 110 (e.g., four struts 110A, 110B, 110C, 110D) can span between adjacent rings 120, as is illustrated in the perspective views of device 100 in FIGS. 3-5. In certain implementations, e.g., as shown in FIGS. 3-5, each strut 110 can have a distinct, corresponding adjustment element 130 (e.g., four adjustment elements 130A, 130B, 130C, 130D) that enable adjustment of individual strut(s) 110 with a modular driving element 160 (FIG. 2), with individual driving elements labeled as 160A, 160B, 160C, 160D in FIGS. 3-5. According to some implementations, a plurality of modular driving elements 160 (FIG. 2) can be used to selectively couple to each of the distinct adjustment elements 130 in the multi-strut configuration. For example, a modular driving element 160 can be used to selectively couple to each adjustment element (e.g., adjustment elements 130A, 130B, 130C, 130D), e.g., at distinct times, or a plurality of distinct modular driving elements 160 (not shown) can be used to selectively couple to each adjustment element 130 at the same time. In certain cases, the modular driving elements 160 are configured to couple with a portion of the ring 120 adjacent to the adjustment element 130, e.g., to fix the modular driving element 160 for actuation. As shown in the example of FIGS. 3-5, the driving element 160 and ring(s) 120 can be connected by a coupler 162, e.g., a fastener, screw, bolt, pin, etc. through complementary mating features (e.g., openings, mounts, etc.).

In certain implementations, a modular driving element 160 is configured to adjust the length of a corresponding strut (or subset of struts) 110 without the presence of all modular driving elements 160 in a set. For example, a given modular driving element 160 can be used to individually adjust the length of a corresponding strut 110 without a driving element 160 for each of the struts 110. In some cases, modular driving elements 160 not required for a particular adjustment can be removed (i.e., decoupled from corresponding adjustment element 130), leaving only those driving elements 160 needed to perform the adjustment between portions of rings 120.

FIGS. 2-5 illustrate one example of an adjustment element 130 and a corresponding (but not limiting) type of modular driving element 160 for a given strut 110. In this example configuration, the modular driving element 160 includes a driving actuator 190 for interfacing with the driven mechanism 180 on the adjustment element 130. In this particular example, the driving actuator 190 includes at least one gear (or other interlocking member configuration) with teeth for interfacing with corresponding teeth on the adjustment element 130. However, in other configurations, the driving actuator 190 can include a clamp, vice, grip, adhesive, etc., that can be connected with the driven mechanism 180 to drive the adjustment element 130. In additional implementations, the driving actuator 190 and the driven mechanism 180 include complementary threads, such that rotating the driving actuator 190 rotates a set of threads and engages complementary threads on the driven mechanism 180. It is understood that where threaded actuators are illustrated, geared actuators could be substituted in certain implementations. In still further implementations, the driving actuator 190 includes magnets and/or pneumatics for engaging with the driven mechanism 180. As described herein, the driving actuator 190 and driven mechanism 180 can include complementary actuators for driving the adjustment element 130. In particular cases, the modular driving element 160 further includes a control unit 200 (FIGS. 2, 6, 8, and 10) for controlling actuation of the driving actuator 190. In certain cases, the control unit 200 includes one or more microcontrollers and/or programmable control circuits for controlling the driving actuator 190. In various implementations, the control unit 200 can include one or more printed circuit boards (PCBs), a driver and a communications module. In certain cases, these components are physically separated, while in other cases they are contained in a common module. In a particular example, the control unit 200 includes a microcontroller configured to initiate rotation of a shaft coupled with a gear mechanism in the driving actuator 190. In certain examples, the modular driving element 160 further includes a motor 210 coupled with the control unit 200 (e.g., via a driver) and the actuator 190, e.g., to drive the actuator 190 in response to a command from the control unit 200. According to various implementations, the modular driving element 160 includes an interface such as an interface button, switch, etc., that is coupled with the control unit 200 and enables actuation of a control command at the control unit 200.

According to some implementations, the control unit 200 and/or driving actuator 190 are coupled to a power source 220 (FIGS. 2, 6, and 8) for powering operation thereof. The power source 220 can include a hard-wired power connection in certain cases. In other cases, the power source 220 includes a battery, enabling portable (wireless) powering of the modular driving element 160. According to particular implementations, the output of power source 220 is prescribed according to an amount of potential adjustment of the length of a given strut 110. That is, the power output for a given modular driving element 160 can vary relative to another modular driving element 160, e.g., where distinct adjustment ranges are desirable for distinct struts 110. In these implementations, a modular driving element 160 with a lower output power source 220 can be connected with a strut 110 (via corresponding adjustment element 130) that requires less adjustment, while a modular driving element 160 with a comparatively higher output power source 220 can be connected with a strut 110 (via corresponding adjustment element 130) that requires less adjustment. In such cases, e.g., where the power source 220 includes an onboard battery (or batteries), a smaller battery can be designated for lesser power output. In certain of these cases, the overall size of the modular driving elements 160 may vary, in part based on the size of those onboard power sources 220 (e.g., batteries).

According to particular implementations, each modular driving element 160 in a given device 100 can have a separate power source 220. For example, each modular driving element 160 can have a separate battery. In particular examples, the separate power source(s) 220 include one or more onboard batteries. According to certain implementations, these onboard batteries can be replaced and/or recharged on an individual basis to enable a medical professional and/or patient to respond to dynamic power needs of the modular driving elements 160. In additional implementations, two or more of the modular driving elements 160 share a common power source. For example, two or more modular driving elements 160 in a device can be coupled to a common battery or a hard-wired (e.g., DC or AC) power source, such as via an external power connector.

The modular driving element 160 can further include a communication system coupled with the control unit 200 (e.g., via wireless or hard-wired means), or integral with the control unit 200, for receiving commands to control (e.g., actuate) the modular driving element 160. The communication system can include a number of hard-wired and/or wireless communication systems, with certain wireless systems configured to communicate over Bluetooth, Bluetooth Low Energy (BLE), radio frequency (RF), Wi-Fi, and/or ultrasound. In additional implementations, the communication system can include an independent subscriber identity module (SIM) assigned to each modular driving element 160. In further cases, the communication system is configured to communicate wirelessly with a remote control system and/or data gathering/analysis platform, e.g., via a cloud-based communication protocol. In particular cases, in response to receiving an actuation command via the communication system, the control unit 200 sends a command to the motor 210, triggering movement (e.g., rotation) of the shaft (e.g., initiation of movement, ceasing of movement, or change in movement) and as an extension, the driving actuator 190. Where the driving actuator 190 is coupled with the driven mechanism 180 on the adjustment element 130, rotation of the driving actuator 190 causes movement of the driven mechanism 180 and the corresponding adjustment at a given strut 110.

In particular cases, each modular driving element 160 is individually programmable to control an amount of the adjustment of the length of each strut 110 to which it is couplable. For example, modular driving elements described herein (e.g., modular driving element 160) may each include a control unit 200 that can be individually programmable to control the amount of adjustment of the length of one or more struts 110. In certain cases, distinct modular driving elements 160 in a given device 100 can be programmed to adjust struts 110 to different lengths, on particular schedules, and/or in particular orders.

In still further implementations, the control unit 200 can be replaced with, or otherwise communicate with (e.g., via communication system) a controller 240 that is external to the body of the modular driving element 160. According to some implementations, controller 240 can provide a means to communicate control commands to the control unit 200 onboard a modular driving element 160 (and receive feedback from onboard electronics). In particular cases, the controller 240 can enable wireless communication of control commands to the control unit 200 and/or feedback data about adjustments from the control unit 200 back to the controller 240, e.g., over any wireless communication protocol described herein. In particular cases, a single controller 240 can be configured to control a set of two or more modular driving elements 160. In some cases, the controller 240 includes a dedicated remote control device for communicating with the modular driving element 160. In additional cases, the controller includes a smart device (e.g., smart phone, smart watch, tablet, etc.) configured to operate a control platform for adjusting the length of a given strut 110. In these instances, the control platform can include a software application (or “app”) configured to execute or otherwise run at the controller 240 for enabling control of one or more modular driving elements 160. According to certain implementations, the control platform enables control functions for one or more modular driving elements 160 from a remote physical location relative to device 100. For example, the control platform can enable connection (e.g., network-based and/or cloud-based connection) between the device 100 and a remote user such as a medical professional. The control platform can enable the remote user to make adjustments to the device 100 via the modular driving elements 160, schedule adjustments via the modular driving elements 160, and/or receive feedback on adjustments, component health/status, etc. without requiring in-person interaction with the device 100.

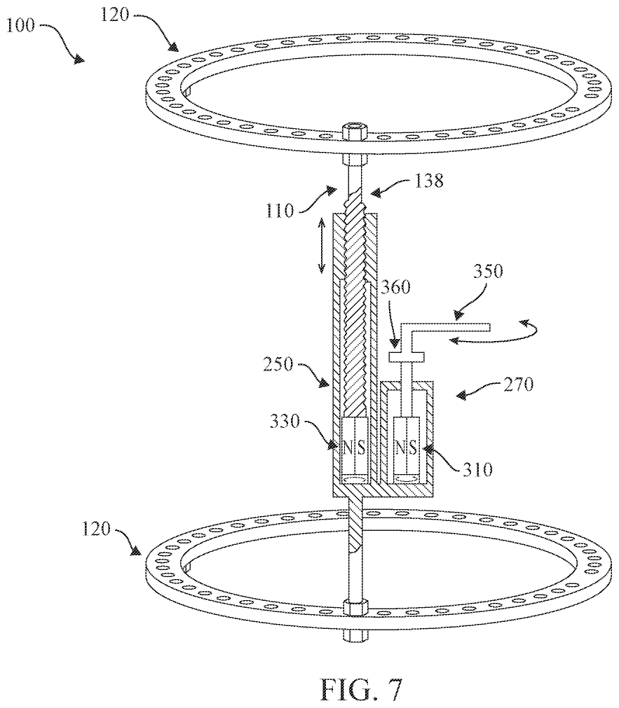

FIG. 6 illustrates additional embodiments of an adjustment element 250 and corresponding modular driving element 260. FIG. 7 illustrates a manual adjustment tool 270 for the adjustment element 250 in FIG. 6 according to various additional embodiments. In certain cases, each of the adjustment element 250 and the modular driving element 260 include magnets configured to interface with one another and enable adjusting of the length of the strut 110. Similar to the driving mechanism for the modular driving element 160, the modular driving element 260 can be actuated via a command received at the control unit 200. In response to a control command, the control unit driver actuates a motor 290 to drive an actuator 300 for at least one magnet 310. In some cases, the actuator 300 includes a gear assembly or another rotary assembly 560 with a corresponding shaft 320 on which the magnet(s) 310 are mounted. In this configuration, a command at the control unit 200 drives the motor 210, and in turn, the actuator 300 and the magnet(s) 310. Corresponding magnets 330 are located in the adjustment element 250, and are coupled with a translatable chamber 340 coupled to the shaft 138 of strut 110.

FIG. 7 illustrates the manual adjustment tool 270, including a crank 350 or other rotary mechanism coupled with a shaft 360 and magnet(s) 310. In a manual adjustment mode, the tool 270 can be used alternatively to the modular driving element 260 to actuate the strut 110 via magnets 330. For example, a user can position the manual adjustment tool 270 adjacent to the adjustment element 250, and turn the crank 350 to rotate the magnet(s) 310, thereby inducing a magnetic field that actuates movement of magnets 330 coupled with the strut 110.

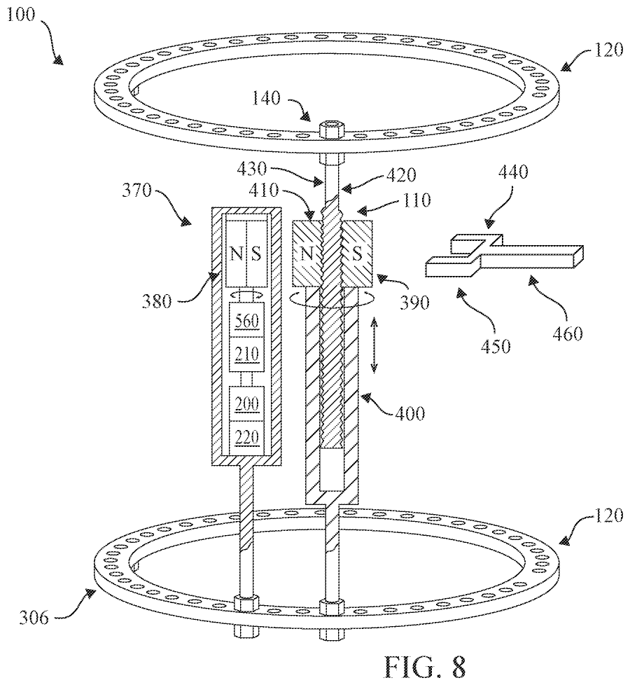

FIG. 8 illustrates an additional implementation of a modular driving element 370 including a set of magnets 380 that can be actuated in a similar manner as the modular driving element 160 in FIG. 2. In contrast to the modular driving element 160, the modular driving element 370 in FIG. 8 uses the set of magnets 380 (e.g., one, two, or more magnets) to actuate movement of a corresponding set of magnets 390 in an adjustment element 400 on the strut 110. As described herein relative to FIG. 2, the control unit 200 can include a microcontroller configured to initiate rotation of a shaft coupled with a gear mechanism in the driving actuator 190. The modular driving element 370 includes motor 210 coupled with the control unit 200 (e.g., via a driver) and the actuator 190, e.g., to drive the actuator 190 in response to a command from the control unit 200. In the implementation depicted in FIG. 8, the strut 110 can include magnets 390 that are coupled with a set of teeth, gears or threads 410. These teeth (gears or threads) 410 are positioned to interface with complementary teeth, gears or threads 420 on a shaft 430 of the strut 110. In this case, changing the position of magnets 380 modifies the magnetic field in which the magnets 390 sit, and forces those magnets 390 in turn to change position (e.g., rotate about the shaft 430). The interaction of the teeth, gears or threads 410, 420 thereby causes translation of the shaft 430, and a corresponding change in relative position of the rings 120. FIG. 8 also illustrates an additional manual adjustment tool 440, which can be used to actuate the magnets 390 by changing the magnetic field in which they sit. For example, the manual adjustment tool 440 can include one or more magnets 450 (e.g., coupled with a handle 460) that can be placed adjacent to the adjustment element 400 and actuated to trigger translation of the shaft 430 (and adjustment of rings 120).

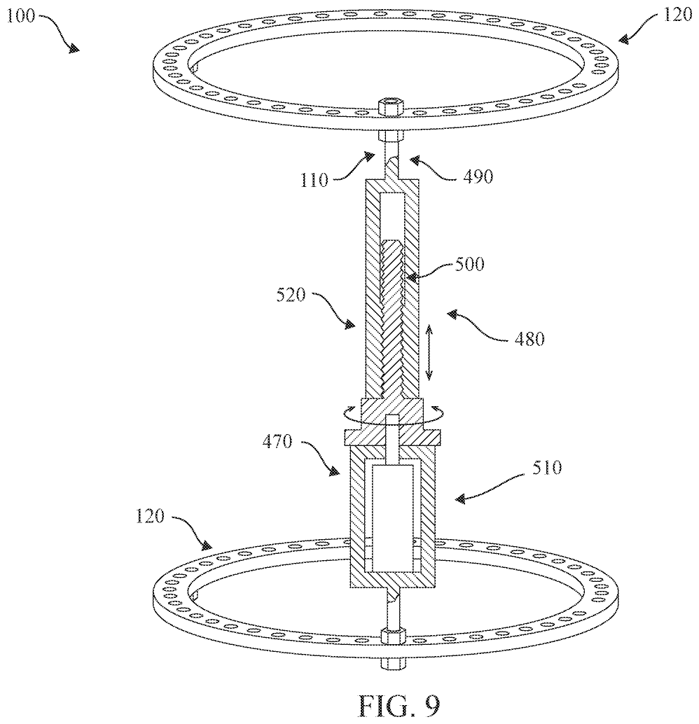

FIG. 9 illustrates an additional implementation of a manual adjustment tool 470 that can be coupled with an adjustment element 480 on a given strut. In certain cases, a strut can be composed of an adjustment element 480 and a distraction rod 490 that are coaxially aligned, such that actuation of the adjustment element 480 causes translation of the distraction rod 490 by forcing displacement of the distraction rod 490 from within the housing 500 of the adjustment element 480. In certain cases, the manual adjustment tool 470 can include a pneumatic driver 510 configured to align with a geared driver or pneumatic driver 520 in the adjustment element 480. According to various implementations, the user can couple the manual adjustment tool 470 coaxially with the adjustment element 480 (e.g., at an end of the housing 500) and actuate the pneumatic driver 510 (e.g., via an interface command such as button push, lever pull, switch flip, etc.) to initiate displacement of the distraction rod 490 (and adjustment of rings 120).

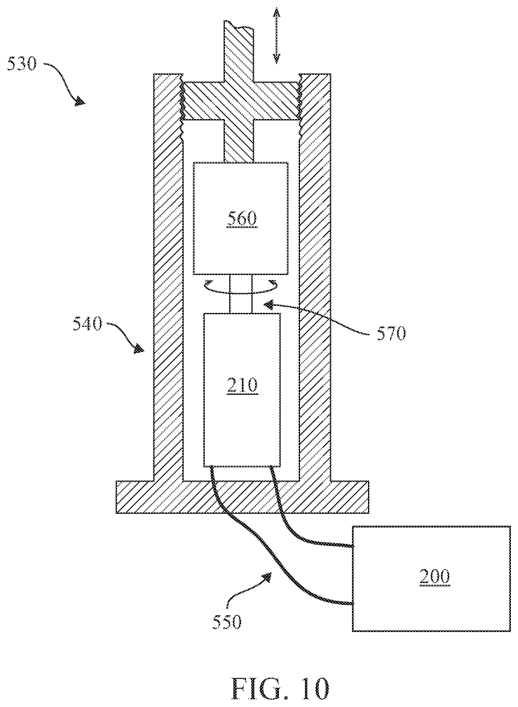

FIG. 10 illustrates an additional modular driving element 530, which can be used interchangeable with the manual adjustment tool 470 (FIG. 9) to interface with the adjustment element 480. In this example, a control unit 200 (e.g., including a PCB and power source) is connected external to the housing 540 of the modular driving element 530, e.g., via a wire 550. In this case, the modular driving element 530 can be coupled with an end of the housing 500 of the adjustment element 480 (FIG. 9), e.g., coaxially, to drive the distraction rod 490. In the depicted example in FIG. 10, gears 560 and a corresponding shaft 570 are used to drive output of motor 210. However, this mechanism can be replaced with a pneumatic driver in various implementations.

In various implementations, one of the adjustment elements described herein (e.g., adjustment element 130, adjustment element 250, adjustment element 400, adjustment element 480) is embedded in the strut 110. For example, in particular cases, the adjustment element is hermetically sealed in the strut 110. In certain of these cases, a power source such as a battery is also hermetically sealed in the strut 110.

In all implementations described herein, the device 100 can further include a feedback system in communication with one or more of the modular driving elements (e.g., modular driving element(s) 160, modular driving element(s) 260, modular driving element(s) 370, modular driving element(s) 530). In certain cases, the modular driving element can provide feedback on a force response to the adjustment of the length of a given strut (e.g., strut 110, FIG. 2). In certain cases, the feedback system includes a sensor onboard the modular driving element, e.g., a sensor that is integrated with or coupled with a control unit 200. In other cases, sensor(s) are external to the housing of the modular driving element, e.g., mounted on a distinct portion of the device 100 and/or located in another portion of the environment in which the device 100 is used (such as an operating room). Non-limiting examples of sensors can include a load cell, a piezo (piezoelectric) sensor, or an imaging sensor (e.g., optical sensor such as a camera, or an ultrasound sensor). Additional sensors that can be integrated in, or otherwise form part of the feedback system can include position and/or speed sensors (e.g., gyroscope/magnetometer, or inertial measurement unit (IMU)), temperature sensors and/or humidity sensors. In certain cases, the feedback system provides instructions to the controller (e.g., control unit 200) to modify a force applied during the length adjustment process for a given strut (e.g., strut 110) based on the feedback on the force response. For example, where the force response feedback indicates that additional adjustment is necessary to achieve a desired displacement of the rings 120, the feedback system can instruct the control unit 200 to modify actuation of the modular driving element. In additional examples, where the force response feedback indicates that too much force is being applied to rings 120 (i.e., the speed of displacement is too fast), the feedback system can instruct the control until 200 to modify actuation of the modular driving element.

In still further implementations, the sensor(s) in the feedback system described herein can be configured to provide data about a load exerted by the modular driving element(s) on an adjustment element, and/or a load exerted by the adjustment element on the strut and/or ring. The sensor(s) can also provide data about a tensile load between components in the modular driving element, adjustment element, strut and/or ring(s). In certain implementations, both torque and compression data are recorded by sensor(s) and provided to the feedback system for analysis and/or action (e.g., to adjust adjustment instructions). It is understood that torque and/or compression data detected by sensors, can represent an inferred or correlated indicator of the torque and/or compression applied to a device or component not physically in contact with the sensor. For example, the sensor on an instrument can be configured to detect torque at the instrument, while that torque is being translated to a driven element in contact with the distal end of the instrument. Similarly, the sensor on an instrument can detect compression at the instrument, while that compression is being translated to an external component, e.g., a driven element.

In additional implementations, one or more device 100 components described herein, e.g., adjustment elements 130, modular driving elements 160, etc. can be communicatively coupled with a navigation system that is configured to detect a position of the instrument(s). In one example, the control unit 240 can include or otherwise communicate with a navigation system in order to provide navigation information about a position of instruments. For example, the navigation system can include an optical tracking system such as a camera or laser-based tracking system, a Global Positioning System (GPS), an inertial measurement unit (IMU), etc. In certain cases, the navigation system is configured to determine a distance moved by the instrument when the instrument changes position, which the navigation system communicates to the control unit 240 (e.g., for processing by the feedback system). One or more components of a navigation system can be located within or otherwise integrated with a housing that is mounted to or otherwise coupled with one or more of the device components.

In certain cases, the feedback system, or functions thereof, can be integrated into the control unit 200 and/or a controller 240 as described herein. In particular cases, the feedback system is part of a software application and is configured to determine what, if any, force adjustment should be made at a given strut 110 based on the force feedback. In some examples, the feedback system includes a model that correlates force response and force applied during adjustment of the length of a strut 110. The model can be based at least in part on historical data from a set of struts in distinct bone fixation devices, e.g., similar to device 100. According to various implementations, the model can be updated periodically, or on a continuous basis, to provide additional data about force response as compared to force applied in one or more struts 110. In certain cases, a version of the model can be downloaded or otherwise stored locally at one or more control units 200 and/or controllers 240 and periodically updated, e.g., via a cloud-based or other network-based software update. This approach can reduce the computational and/or storage requirements at control unit(s) 200 and controller(s) 240 that may be local to the device 100.

In additional implementations, the feedback system is configured to provide post-operative data, post-adjustment data, and analysis of alignment procedure and/or device usage, e.g., to enhance future procedures and/or diagnose inefficiencies in a past procedure. In certain implementations, the feedback system is configured to update the control instructions for control unit(s) 210 based on identified inefficiencies or errors in fixator sequencing and/or device usage during/after a given procedure. In particular implementations, the feedback system includes a logic engine configured to modify instructions iteratively, e.g., on a procedure-by-procedure or patient-by-patient basis.

Various additional aspects of the disclosure can include a method of adjusting a bone fixation device. Using FIGS. 1 and 2 strictly for the simplicity of illustration, the method can include adjusting a bone fixation device (e.g., bone fixation device 100) by: (i) coupling a modular driving element (e.g., modular driving element 160) or a manual adjustment tool (e.g., manual adjustment tool 170) to an adjustment element (e.g., adjustment element 130). As described herein, the modular driving element or manual adjustment tool can be configured to mechanically couple with the adjustment element and/or mount adjacent to the adjustment element, such as on rings 120. The method can further include: (ii) actuating adjustment of the length of the strut (e.g., strut 110) with the modular driving element or the manual adjustment tool. In certain cases, after adjusting the length of the strut 110, a method can further include: (iii) decoupling the coupled modular driving element (e.g., modular driving element 160) or manual adjustment tool (e.g., manual adjustment tool 170) from the adjustment element (e.g., adjustment element 130). After decoupling the driving element or manual adjustment tool, the method can further include: (iv) coupling the other one of (a) the modular driving element or (b) the manual adjustment tool to the adjustment element, and (v) actuating adjustment of the length of the strut 110.

In certain cases, a method can include imaging a bone connected with the bone fixation devices (e.g., device 10) described and illustrated herein. For example, a method can include: (I) decoupling a (previously coupled) modular driving element (e.g., modular driving element 160) from an adjustment element (e.g., adjustment element 130), and (II) imaging the bone with Mill and/or X-ray imaging after decoupling the modular driving element from the adjustment element. After imaging, the method can further include: (III) either (a) coupling (or recoupling) the modular driving element to the adjustment element, or (b) coupling a manual adjustment tool (e.g., manual adjustment tool 170) to the adjustment element 130.

As noted herein, the bone fixation devices disclosed according to various implementations provide numerous benefits relative to conventional bone fixation devices. For example, the bone fixation devices disclosed according to various implementations can enable modular, adaptive adjustment of bone fixators in both operative and post-operative settings. Further, the bone fixation devices disclosed according to various implementations can be remotely controlled and/or monitored, enhancing patient outcomes by improving medical professional involvement in recovery. Even further, the bone fixation devices disclosed according to various implementations enable effective imaging of bone recovery, thereby saving time and reducing complications while monitoring the progress of a fixation process.

The functionality described herein, or portions thereof, and its various modifications (hereinafter “the functions”) can be implemented, at least in part, via a computer program product, e.g., a computer program tangibly embodied in an information carrier, such as one or more non-transitory machine-readable media, for execution by, or to control the operation of, one or more data processing apparatus, e.g., a programmable processor, a computer, multiple computers, and/or programmable logic components.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a network.

Actions associated with implementing all or part of the functions can be performed by one or more programmable processors executing one or more computer programs to perform the functions of the calibration process. All or part of the functions can be implemented as, special purpose logic circuitry, e.g., an FPGA and/or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. Components of a computer include a processor for executing instructions and one or more memory devices for storing instructions and data.

In various implementations, components described as being “coupled” to one another can be joined along one or more interfaces. In some implementations, these interfaces can include junctions between distinct components, and in other cases, these interfaces can include a solidly and/or integrally formed interconnection. That is, in some cases, components that are “coupled” to one another can be simultaneously formed to define a single continuous member. However, in other implementations, these coupled components can be formed as separate members and be subsequently joined through known processes (e.g., soldering, fastening, ultrasonic welding, bonding). In various implementations, electronic components described as being “coupled” can be linked via conventional hard-wired and/or wireless means such that these electronic components can communicate data with one another. Additionally, sub-components within a given component can be considered to be linked via conventional pathways, which may not necessarily be illustrated.

While inventive features described herein have been described in terms of preferred embodiments for achieving the objectives, it will be appreciated by those skilled in the art that variations may be accomplished in view of these teachings without deviating from the spirit or scope of the invention. Also, while this invention has been described according to a preferred use in spinal applications, it will be appreciated that it may be applied to various other uses desiring surgical fixation, for example, the fixation of long bones.

A number of implementations have been described. Nevertheless, it will be understood that additional modifications may be made without departing from the scope of the inventive concepts described herein, and, accordingly, other implementations are within the scope of the following claims.

We claim:

1. A bone fixation device for attaching to a bone externally to a body, the bone fixation device comprising:
a set of two or more rings, each ring in the set of rings being coupled with distinct portions of the bone via corresponding bone connectors;
a strut spanning between two of the rings in the set of rings;
an adjustment element for adjusting a length of the strut; and
a modular driving element for selectively coupling to the adjustment element and controlling adjustment of the length of the strut.

2. The bone fixation device of claim 1, wherein the adjustment element is fixed to the strut and the modular driving element is couplable and decouplable from the adjustment element.

3. The bone fixation device of claim 1, further comprising a plurality of additional struts spanning between the two rings, and wherein each of the plurality of additional struts comprises a distinct adjustment element for adjusting a length of the corresponding strut.

4. The bone fixation device of claim 3, further comprising a plurality of additional modular driving elements for selectively coupling to each of the distinct adjustment elements to control adjustment of the length of each corresponding strut, and wherein:
a) each of the modular driving elements is individually programmable to control an amount of the adjustment of the length of the corresponding strut,
b) at least one of the modular driving elements is configured to adjust the length of the corresponding strut without the presence of all of the modular driving elements,
c) each of the modular driving elements has a separate power source, or
d) at least two of the modular driving elements share a common power source.

5. The bone fixation device of claim 1, wherein the set of rings comprises at least three rings.

6. The bone fixation device of claim 1, wherein the adjustment element includes a linear actuator.

7. The bone fixation device of claim 1, wherein the modular driving element is wirelessly coupled with a controller for controlling adjustment of the length of the strut or is wirelessly coupled with the controller for controlling adjustment of the length of the strut, and wherein:
a) the controller comprises a remote control device dedicated to controlling adjustment of the length of the strut or a smart device configured to operate a control platform for adjusting the length of the strut, or
b) the controller is configured to run as a control platform at a remote location relative to the bone fixation device.

8. The bone fixation device of claim 1, wherein the adjustment element and the modular driving element each comprise:
a) a complementary linkage configured to interface with one another and enable adjusting the length of the strut, or
b) magnets configured to interface with one another and enable adjusting the length of the strut.

9. The bone fixation device of claim 1, wherein the modular driving element comprises:
a driving actuator for interfacing with a driven mechanism on the adjustment element;
a control unit for controlling actuation of the driving actuator;
a communication system coupled with the control unit, the communication system configured to receive instructions to actuate the driving actuator; and
at least one of:
a) a motor coupled with the control unit and the driving actuator, or
b) a power source coupled with the control unit and the driving actuator, wherein an output of the power source is prescribed according to an amount of potential adjustment of the length of the strut.

10. The bone fixation device of claim 1, wherein the adjustment element is embedded in the strut.

11. The bone fixation device of claim 1, wherein the bone fixation device provides negligible distortion of imaging of the bone under both X-ray imaging and MM.

12. The bone fixation device of claim 1, further comprising:
a feedback system in communication with the modular driving element, wherein the modular driving element provides feedback on a force response to the adjustment of the length of the strut.

13. The bone fixation device of claim 12, wherein the feedback system comprises a sensor, and either:
a) the feedback system provides instructions to a controller for the modular driving element to modify a force applied during the adjustment of the length of the strut based on the feedback on the force response, or
b) the feedback system comprises a model correlating force response and force applied during adjustment of the length of the strut, wherein the model is based at least in part on historical data from a set of struts in distinct bone fixation devices.

14. The bone fixation device of claim 1, further comprising a manual adjustment tool for mating with the adjustment element to adjust the length of the strut while the modular driving element is decoupled from the adjustment element.

15. The bone fixation device of claim 1, wherein the bone connectors include at least one of bone screws or wires.

16. A method of adjusting a bone fixation device attached to a bone external to a body, the bone fixation device having: a set of at least two rings coupled with distinct portions of the bone via corresponding bone connectors; at least one strut spanning between two of the rings in the set of rings; and an adjustment element for adjusting a length of the strut, the method comprising:
coupling a modular driving element or a manual adjustment tool to the adjustment element; and
actuating adjustment of the length of the strut with the modular driving element or the manual adjustment tool.

17. The method of claim 16, wherein the modular driving element comprises a self-powered actuator, the method further comprising:
decoupling the coupled modular driving element or manual adjustment tool from the adjustment element;
coupling the other one of the modular driving element or the manual adjustment tool to the adjustment element; and
actuating adjustment of the length of the strut.

18. The method of claim 16, further comprising:
for the modular driving element: receiving feedback about a force applied in actuating the adjustment of the length of the strut; and
adjusting the force applied in actuating adjustment of the length of the strut in response to the feedback indicating that the force applied in actuating adjustment of the length of the strut deviates from a threshold.

19. A method of imaging a bone connected with a bone fixation device external to a body, the bone fixation device having: a set of at least two rings coupled with distinct portions of the bone via corresponding bone connectors; at least one strut spanning between two of the rings in the set of rings; an adjustment element for adjusting a length of the strut; and a modular driving element coupled to the adjustment element, the method comprising:
decoupling the modular driving element from the adjustment element; and
imaging the bone with at least one of MRI or X-ray imaging.

20. The method of claim 19, wherein when the modular driving element is decoupled from the adjustment element, the bone fixation device provides negligible distortion of imaging of the bone under MM and X-ray imaging,
the method further comprising coupling the modular driving element to the adjustment element or coupling a manual adjustment tool to the adjustment element after imaging the bone.
